(19) United States
(12) Patent Application Publication
(10) Pub. No.: US 2008/0226576 A1
(43) Pub. Date: Sep. 18, 2008

(54) Hair shaping compositions comprising at least one silicone and at least one alkoxysilane having solubilizing functional groups

(76) Inventors: Katarina Benabdillah, le Plessis-Bouchard (FR); Patrice Lerda, Paris (FR); Isabelle Rollat-Corvol, Paris (FR); Henri Samain, Bievres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/003,101

(22) Filed: Dec. 20, 2007

Related U.S. Application Data
(60) Provisional application No. 60/907,308, filed on Mar. 28, 2007.

(30) Foreign Application Priority Data
Dec. 20, 2006 (FR) 0655758

(51) Int. Cl. A61K 8/72; A61K 8/25; A61K 8/40; A61Q 5/06

(52) U.S. Cl. 424/70.9; 424/70.1; 424/70.122

(57) ABSTRACT

Disclosed herein is a method for preventing the deterioration of the feel of the hair upon repeated application of a hair shaping composition comprising at least one alkoxysilane having at least one solubilizing functional group, wherein the method comprises applying to the hair said shaping composition wherein said composition further comprises at least one silicone.

This application claims benefit of U.S. Provisional Application No. 60/907,308, filed Mar. 28, 2007, the contents of which are incorporated herein by reference. This application also claims benefit of priority under 35 U.S.C. § 119 to French Patent Application No. FR 06 55758, filed Dec. 20, 2006, the contents of which are also incorporated herein by reference.

Disclosed herein is a method for preventing deterioration of the feel of the hair upon repeated application of a hair shaping composition comprising at least one alkoxysilane comprising at least one solubilizing functional group, said method comprising applying to the hair said hair shaping composition, wherein said hair shaping composition further comprises at least one silicone. Also disclosed herein are cosmetic compositions comprising at least one silicone and at least one alkoxysilane comprising at least one solubilizing functional group.

People with thin and frizzed hair are seeking long-lasting hair styling effects that would add weight, body, and extra-volume to the hair.

Hair products enable a method to non-permanently reshape the hair and to obtain such hair styling effects. They may be applied onto wet or dry hair, prior to manually styling the hair or using a brush or a comb. They typically comprise at least one cosmetic active agent, such as fixing polymers, thickeners, glycerol, silicones, and waxes.

Once they have been applied onto the hair and been dried, these products may significantly harden. They thus may produce a full-bodied and dry feel required for preserving the hair shape and volume.

Such hair products also may suffer from giving styling effects to the hair that can disappear after the first shampoo following the hair product application. They hence have to be applied every day.

However, there are some hair products that can enable obtaining such hair styling effects, for example, giving weight, body, and extra-volume, whilst resisting to several shampoos.

It is known, for example, from French Patent Nos. FR 2 783 164, FR-2 783 167, FR 2 783 165, and FR 2 798 063 to use silicon organic compounds chosen from silanes comprising one silicon atom and siloxanes comprising two or three silicon atoms, these compounds being water-soluble, for preserving the hair style or the hair shape. Such compounds may make it possible to obtain hair styling effects, for example, to add weight and/or body to the hair, to make detangling easier, and to refine curl shape, while being resistant to several shampoos. However, these effects can be relatively poor and irregular and can become indistinct after about two weeks.

Cosmetic compositions are also described, for instance, in French Patent Nos. FR 552 855, FR 552 853, and FR 552 856, said compositions comprising silicon organic compounds that are soluble in alcohol-based media or in water-alcoholic-type media containing hydrolyzable and polymerizable functional groups, these compositions being able to obtain strong hair styling effects that may last throughout several shampoos.

However, after a repeated application of the hair products comprising silicon organic compounds, especially solubilizing functional group-containing alkoxysilanes, the hair may become dry and coarse, and may have a sensitized feel and/or be damaged.

Therefore, there is a need in the art for a cosmetic composition for hair shaping that enables the user to obtain strong styling effects lasting throughout several shampoos while keeping a natural hair feel.

The present inventors have found, surprisingly, that including at least one silicone in a hair shaping composition comprising at least one alkoxysilane comprising at least one solubilizing functional group can make it possible to prevent deterioration of the feel of the hair upon repeated application of said hair shaping composition.

Thus disclosed herein is a method for preventing deterioration of the feel of the hair upon repeated application of a hair shaping composition comprising at least one alkoxysilane comprising at least one solubilizing functional group, the method comprising applying to the hair said hair shaping composition, wherein said shaping composition further comprises at least one silicone.

As used herein, the term “preventing deterioration of the feel of the hair,” means preventing the hair from becoming dry and/or coarse, from having a sensitized hair feeling, and from being damaged.

Alkoxysilanes Comprising at Least One Solubilizing Functional Group

As used herein, the term “at least one solubilizing functional group” means any functional chemical group facilitating the bringing into solution of the alkoxysilane in the solvent or in a combination of solvents of the composition, for example, in solvents chosen from water and water-alcoholic mixtures.

Suitable solubilizing functional groups for use in accordance with the present disclosure include, but are not limited to, primary, secondary, and tertiary amine, aromatic amine, alcohol, carboxylic acid, sulfonic acid, anhydride, carbamate, urea, guanidine, aldehyde, ester, amide, epoxy, pyrrole, dihydroimidazole, gluconamide, pyridyle, and polyether groups.

The at least one alkoxysilane present in the composition comprises at least one solubilizing functional group, which may be identical or different, such as those previously defined.

The at least one alkoxysilane comprising at least one solubilizing functional group present in the composition of the present disclosure may comprise at least one silicon atom, for example, one silicon atom.

The at least one alkoxysilane comprising at least one solubilizing functional group present in the composition may, in at least one embodiment, comprise two or three alkoxy functions. In another embodiment, the alkoxy functional groups are chosen from methoxy and ethoxy functional groups.

According to a further embodiment, the at least one alkoxysilane comprising at least one solubilizing functional group present in the composition of the present disclosure is chosen from compounds of formula (I):

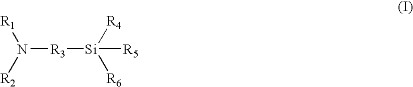

wherein:

R₄ is chosen from halogen atoms, OR′ groups, and R₁₁ groups;

R₅ is chosen from halogen atoms, OR″ groups, and R₁₂ groups;

R₆ is chosen from halogen atoms, OR′″ groups, and R₁₃ groups;

R₁, R₂, R₃, R′, R″, R′″, R₁₁, R₁₂, and R₁₃, which may be identical or different, are chosen from linear and branched, saturated and unsaturated hydrocarbon groups, optionally bearing at least one additional chemical group, wherein R₁, R₂, R′, R″, and R′″ may also be chosen from hydrogen; at least two groups R₄, R₅, and R₆ are different from R₁₁, R₁₂, and R₁₃, and at least two groups R′, R″, and R′″ are not hydrogen.

In at least one embodiment, the R₁, R₂, R′, R′₁, R′₂, R′₃, R″, and R′″ groups are chosen from C₁-C₁₂ alkyl, C₆-C₁₄ aryl, C₁-C₈ alkyl-C₆-C₁₄ aryl, and C₆-C₁₄ aryl-C₁-C₈-alkyl radicals.

According to a second embodiment of the present disclosure, the at least one alkoxysilane comprising at least one solubilizing functional group present in the composition is chosen from compounds of formula (II):

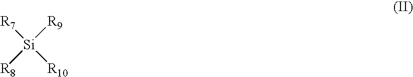

wherein:

R₉ is chosen from halogen atoms and OR′₉ groups and R₁₀ is chosen from halogen atoms and OR′₁₀ groups; wherein at least one of R₉ and R₁₀ is not a halogen;

R′₉ and R′₁₀, which may be identical or different, are chosen from hydrogen, and linear and branched, saturated and unsaturated C₁-C₁₄ hydrocarbon groups; wherein at least one of R₉ and R₁₀ is not hydrogen;

R₇ is a non hydrolyzable functional group providing a cosmetic effect, and

R₈ is a non hydrolyzable functional group bearing at least one function chosen from: amines, carboxylic acids and salts thereof, sulfonic acids and salts thereof, polyols such as glycol, polyethers such as polyalkylene ether, and phosphoric acids and salts thereof.

As used herein, the term “functional group providing a cosmetic effect” means a group derived from an entity chosen from reducing agents, oxidizing agents, coloring agents, polymers, surfactants, antibacterial agents, and UV absorbing filters.

In at least one embodiment, the functional group providing a cosmetic effect is a group derived from a coloring agent.

According to a third embodiment of the present disclosure, the at least one alkoxysilane comprising at least one solubilizing functional group present in the composition of the present disclosure is chosen from compounds of formula (III):

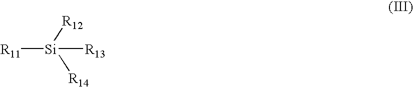

wherein:

R₁₂ is chosen from halogen atoms, OR′₁₂ groups, and R_{O} groups;

R₁₃ is chosen from halogen atoms, OR′₁₃ groups, and R′_{O} groups;

R₁₄ is chosen from halogen atoms, OR′₁₄ groups, and R″_{O} groups;

wherein at least two groups R₁₂, R₁,₃ and R₁₄ are different from R_{O}, R′_{O}, and R″_{O} groups;

R₁₁ is a group chosen from groups bearing at least one function chosen from: carboxylic acids and salts thereof, sulfonic acids and salts thereof, and polyalkylethers; and

Ro, R′o, R″o, R′₁₂, R′₁₃, and R′₁₄, which may be identical or different, are chosen from linear and branched, saturated and unsaturated, C₁-C₁₄ hydrocarbon groups optionally bearing at least one additional chemical functional group chosen from: carboxylic acids and salts thereof, sulfonic acids and salts thereof, and polyalkylether functions, wherein R′₁₂, R′₁₃, and R₁₄ may also be chosen from hydrogen, and wherein at least two of the groups R′₁₂, R′₁₃, and R′₁₄ are not hydrogen.

In at least one embodiment, the R′₁₂, R′₁₃, R′₁₄, R_{O}, R′_{O}, and R″_{O} groups are chosen from C₁-C₁₂ alkyl groups, C₆-C₁₄ aryl groups, C₁-C₈ alkyl-C₆-C₁₄ aryl groups, and C₆-C₁₄ aryl-C₁-C₈ alkyl groups.

According to another embodiment of the present disclosure, the at least one alkoxysilane comprising at least one solubilizing functional group present in the composition of the present disclosure is chosen from compounds of formula (IV):

(R₂₁O)ₓ(R₂₂)_{y}Si-(A)ₚ-[NR₂₃-(A′)_{p′}]_{q}-[NR′₂₃-(A″)_{p″}]_{q′}-Si—(R′₂₂)_{y′}(OR′₂₁)_{x′} (IV)

wherein:

R₂₁, R₂₂, R′₂₁, and R′₂₂, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,

x is an integer ranging from 1 to 3,

y=3-x,

x′ is an integer ranging from 1 to 3,

y′=3-x′,

p=0 or 1,

p′=0 or 1,

p″=0 or 1,

q=0 or 1,

q′=0 or 1,

wherein at least one of q or q′ is not equal to zero,

A, A′, and A″, which may be identical or different, are chosen from linear and branched C₁-C₂₀ alkylene divalent radicals, and

R₂₃ and R′₂₃, which may be identical or different, are chosen from hydrogen and linear and branched, saturated and unsaturated hydrocarbon chains, optionally comprising at least one heteroatom, optionally interrupted by or substituted with at least one entity chosen from: ether, C₁-C₂₀ alcohol ester, amine, carboxyl, alkoxysilane, C₆-C₃₀ aryl, hydroxyl, and carbonyl groups, and aromatic, heterocyclic, and non-heterocyclic rings, optionally substituted with at least one group chosen from C₃-C₂₀ alcohol ester, amine, amide, carboxyl, alkoxysilane, hydroxyl, carbonyl, and acyl groups.

As defined above, R₂₁, R₂₂, R′₂₁, and R′₂₂, which may be identical or different, may be chosen from hydrocarbon chains. As used herein, the term “hydrocarbon chain” means, for example, a chain comprising from 1 to 10 carbon atoms.

Likewise, R₂₃ and R′₂₃ may be chosen from hydrocarbon chains. In such an embodiment, the hydrocarbon chains may comprise from 1 to 10 carbon atoms.

According to one embodiment, the aromatic ring comprises from 6 to 30 carbon atoms. In another embodiment, the aromatic ring is an optionally substituted phenyl radical.

In at least one embodiment, in formula (IV) above:

R₂₁=R′₂₁,

R₂₂=R′₂₂,

x=x′,

y=y′,

p=p′,

A=A′,

q=1, and

q′=0.

According to a further embodiment, the at least one alkoxysilane comprising at least one solubilizing functional group used according to the present disclosure may also have at least one of the following characteristics:

R₂₁, R₂₂, R′₂₁, and R′₂₂, which may be identical or different, are chosen from C₁-C₄ alkyl groups;

p=p′=1;

A and A′, which may be identical or different, are chosen from linear C₁-C₄ alkylene groups; and/or

R₂₃ is hydrogen.

According to this embodiment, the at least one alkoxysilane comprising at least one solubilizing functional group may be chosen from compounds of formula (V):

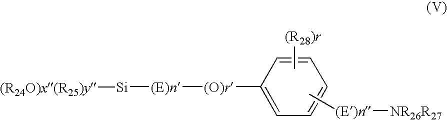

wherein:

R₂₄ and R₂₅, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,

x″=2 or 3,

y″=3-x″,

n″=0 or 1,

n″=0 or 1,

E and E′, which may be identical or different, are chosen from linear and branched C₁-C₂₀ alkylene divalent radicals,

R₂₆ and R₂₇, which may be identical or different, are chosen from hydrogen and linear and branched, saturated and unsaturated hydrocarbon chains, optionally comprising at least one heteroatom, optionally interrupted by or substituted with at least one entity chosen from: ether, C₁-C₂₀ alcohol ester, amine, carboxyl, alkoxysilane, C₆-C₃₀ aryl, hydroxyl, and carbonyl groups, and aromatic, heterocyclic, and non-heterocyclic rings, optionally substituted with at least one group chosen from: C₁-C₂₀ alcohol ester, amine, amide, carboxyl, alkoxysilane, hydroxyl, carbonyl, and acyl groups,

r is an integer ranging from 0 to 4,

r′=0 or 1, and

R₂₈, which may be identical or different, is chosen from hydrogen and linear and branched, saturated and unsaturated hydrocarbon chains, comprising, for example, from 1 to 10 carbon atoms and optionally at least one heteroatom, optionally interrupted by or substituted with at least one entity chosen from: ether, C₁-C₂₀ alcohol ester, amine, carboxyl, alkoxysilane, C₆-C₃₀ aryl, hydroxyl, and carbonyl groups, and aromatic, heterocyclic, and non-heterocyclic rings, optionally substituted with at least one group chosen from: C₁-C₂₀ alcohol ester, amine, amide, carboxyl, alkoxysilane, hydroxyl, carbonyl, and acyl groups.

As defined above, R₂₄ and R₂₅, which may be identical or different, may be chosen from hydrocarbon chains. As used herein, the term “hydrocarbon chain” is intended to mean a chain comprising, for example, from 1 to 10 carbon atoms.

Likewise, R₂₆ and R₂₇ may be chosen from hydrocarbon chains. In this embodiment, the hydrocarbon chains may comprise from 1 to 10 carbon atoms.

According to another embodiment, the aromatic ring comprises from 6 to 30 carbon atoms. In a further embodiment, the aromatic ring is an optionally substituted phenyl radical.

According to at least one embodiment, the at least one alkoxysilane comprising at least one solubilizing functional group used in accordance with the present disclosure may have at least one of the following characteristics:

R₂₄ is a C₁-C₄ alkyl group;

x″=3;

n′=n″=1

r=r′=0; and/or

R₂₆ and R₂₇, which may be identical or different, are chosen from hydrogen and groups chosen from C₁-C₄ alkyl groups, C₁-C₄ hydroxyalkyl groups, and C₁-C₄ aminoalkyl groups.

According to this embodiment, the at least one alkoxysilane comprising at least one solubilizing functional group may be chosen from:

3-(m-aminophenoxy)propyl trimethoxysilane, of formula:

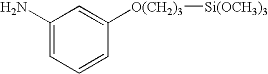

p-aminophenyl trimethoxysilane, of formula:

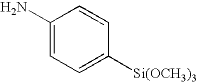

and

N-(2-aminoethylaminomethyl)phenethyl trimethoxysilane, of formula:

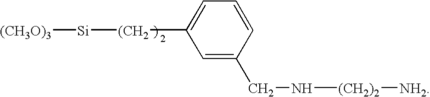

According to a further embodiment of the present disclosure, the at least one alkoxysilane comprising at least one solubilizing functional group present in the composition is chosen from compounds of formula (VI):

(R₂₉O)x₁(R₃₀)y₁-Si-(A₁)ₛ-CH═O (VI)

wherein:

R₂₉ and R₃₀, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,

x₁=2 or 3,

y₁=3-x₁,

A₁ is chosen from linear and branched C₁-C₂₀ alkylene divalent radicals, optionally interrupted by or substituted with at least one group chosen from C₁-C₃₀ alcohol ester, amine, carboxyl, alkoxysilane, C₆-C₃₀ aryl, hydroxyl, and carbonyl groups, and

s=0 or 1.

As defined above, R₂₉ and R₃₀, which may be identical or different can be chosen from hydrocarbon chains. As used herein, the term “hydrocarbon chain” means a chain comprising, for example, from 1 to 10 carbon atoms.

In another embodiment, the at least one alkoxysilane comprising at least one solubilizing functional group may have at least one of the following characteristics:
R₂₉ and R₃₀, which may be identical or different, are chosen from C₁-C₄, alkyl groups;
s=1; and
A₁ is a linear C₁-C₄ alkylene group

According to this embodiment, the at least one alkoxysilane comprising at least one solubilizing functional group may be chosen from:

triethoxysilyl butyraldehyde, of formula:

(CH₃CH₂O)₃—Si—(CH₂)₃—CH═O

triethoxysilyl undecanal, of formula:

(CH₃CH₂O)₃—Si—(CH₂)₁₀—CH═O

and

triethoxysilyl undecanal, ethylene glycol acetal, of formula:

(CH₃CH₂O)₃—Si—(CH₂)₁₀—CH(OCH₂)₂.

In a further embodiment, the at least one alkoxysilane comprising at least one solubilizing functional group is chosen from compounds of formula (VII):

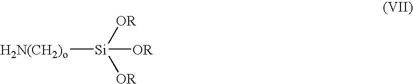

wherein the R radicals, which may be identical or different, are chosen from C₁-C₆ alkyl radicals and n is an integer ranging from 1 to 6, for example, from 2 to 4.

In at least one embodiment, the at least one alkoxysilane comprising at least one solubilizing functional group present in the composition of the present disclosure is a γ-aminopropyl triethoxysilane.

The at least one alkoxysilane comprising at least one solubilizing functional group may be present in the composition in an amount ranging from 0.1 to 20%, such as from 1 to 15% by weight relative to the total weight of the composition.

Silicones

The at least one silicone used according to the present disclosure may be chosen from polydialkyl siloxanes, such as polydimethyl siloxanes (PDMS), polyalkylaryl siloxanes, polydiaryl siloxanes, and organomodified polysiloxanes comprising at least one functional moiety chosen from poly(oxyalkylene) moieties, amine moieties, alkoxy moieties, hydroxylated moieties, acyloxyalkyl moieties, carboxylic acid moieties, hydroxyacylamino moieties, acrylic moieties, polyamine moieties and oxazoline moieties, and silicone-based celluloses.

Silicones suitable for use according to the present disclosure include, but are not limited to, volatile and non volatile, cyclic, linear, and branched silicones, optionally modified with organic moieties, having a viscosity ranging from 5×10⁻⁶ to 2.5 m²/s at 25° C., for example, from 1×0-5 to 1 m²/s.

Silicones that may be used according to the present disclosure may be soluble or insoluble in the composition and may be, for instance, polyorganosiloxanes that are not soluble in the composition of the present disclosure. They may be in a form chosen from fluids, waxes, resins, and gums.

Organopolysiloxanes are defined, for instance, by Walter NOLL in “Chemistry and Technology of Silicones” (1968), Academic Press. They may be volatile or non volatile.

When they are volatile, the silicones may be chosen from those having a boiling point ranging from 60° C. to 260° C., for example:
(i) cyclic polydialkyl siloxanes comprising from 3 to 7, for instance, from 4 to 5 silicon atoms. Non-limiting examples of such siloxanes include the octamethyl cyclotetrasiloxane marketed, for instance, under the trade name VOLATILE SILICONE® 7207 by UNION CARBIDE and SILBIONE® 70045 V2 by RHODIA, the decamethyl cyclopentasiloxane marketed under the trade name VOLATILE SILICONE® 7158 by UNION CARBIDE, and SILBIONE®70045 V5 by RHODIA, as well as mixtures thereof. Cyclomethicones may also be used, for example, those marketed under the references DC 244, DC 245, DC 344, DC 345, and DC 246 by DOW CORNING.

Cyclocopolymers of the dimethyl siloxane/methylalkyl siloxane type may also be used, such as SILICONE VOLATILE® FZ 3109 marketed by UNION CARBIDE, of formula:

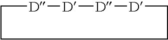

wherein:

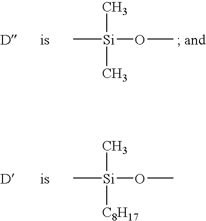

Combinations of cyclic polydialkyl siloxanes with silicon derived organic compounds may also be used, such as the octamethyl cyclotetrasiloxane and tetratrimethylsilyl pentaerythritol (50/50) mixture and the octamethyl cyclotetrasiloxane and oxy-1,1′-(hexa-2,2,2′,2′,3,3′-trimethylsilyloxy) bis-neopentane mixture;

(ii) linear volatile polydialkyl siloxanes comprising from 2 to 9 silicon atoms and having a viscosity equal to or less than 5×10⁻⁶ m²/s at 25° C. A non-limiting example of such a compound is the decamethyl tetrasiloxane marketed, for instance, under the trade name “SH-200” by TORAY SILICONE. Silicones belonging to this class are also described, for example, in Cosmetics and Toiletries, Vol. 91, Jan. 76, P. 27-32—TODD & BYERS “Volatile Silicone fluids for cosmetics.”

In at least one embodiment, the silicones may be chosen from non volatile silicones, such as polydialkyl siloxanes, polyalkylaryl siloxanes, polydiaryl siloxanes, waxes, gums, silicone resins, and polyorganosiloxanes modified with the hereabove organofunctional moieties.

According to another embodiment, the silicones are chosen from polydialkyl siloxanes, for example, polydimethyl siloxanes with trimethylsilyl end groups known under the trade name dimethicones. The viscosity of these silicones is measured at 25° C. according to ASTM 445 standard Appendix C.

Non limiting examples of commercial products corresponding to such polydialkyl siloxanes include:

SILBIONE® fluids of the series 47 and 70 047 and MIRASIL® fluids marketed by RHODIA, for example the 70 047 fluid V 500 000;

fluids of the MIRASIL® series marketed by RHODIA;

fluids of the series 200 marketed by DOW CORNING such as DC200, with a viscosity of 60,000 mm²/s;

VISCASIL® fluids of GENERAL ELECTRIC and some fluids of the SF series (e.g., SF 96 and SF 18) of GENERAL ELECTRIC; and

the fluid marketed under the reference DC 1664 by DOW CORNING.

Polydimethyl siloxanes with dimethyl silanol end groups may also be used, for example, those sold under the trade name dimethiconol (CTFA), such as fluids of the 48 series marketed by RHODIA.

Products marketed under the trade names “ABIL Wax® 9800 and 9801” by GOLDSCHMIDT belonging to this class of polydialkyl siloxanes, that are polydialkyl (C₁-C₂₀) siloxanes may also be used.

Polydimethyl siloxane waxes may also be used.

Silicone gums suitable for use according to the present disclosure include, but are not limited to, polydialkyl siloxanes, such as polydimethyl siloxanes having high number average molecular weights ranging from 200,000 to 1,000,000, alone or as mixtures in a solvent. This solvent may be chosen from volatile silicones, polydimethyl siloxane (PDMS) fluids, polyphenylmethyl siloxane (PPMS) fluids, isoparaffins, polyisobutylenes, methylene chloride, pentane, dodecane, tridecane, and mixtures thereof. Silicone gums may also be chosen, for example, from amodimethicones, such as the products marketed under the references DC 929 Emulsion and DC 939 Emulsion by DOW CORNING, and the Belsil ADM LOG1 product marketed by WACKER.

According to at least one embodiment, combinations of silicones may also be used, such as:

mixtures of a polydimethyl siloxane hydroxylated at the end of the chain, or dimethiconol (CTFA), and a cyclic polydimethyl siloxane also called cyclomethicone (CTFA), such as the Q2 1401 product marketed by DOW CORNING;

mixtures of a polydimethyl siloxane gum and a cyclic silicone, such as the SF 1214 Silicone Fluid product marketed by GENERAL ELECTRIC, such product being a SF 30 gum corresponding to a dimethicone, with a number average molecular weight of 500,000 solubilized in the SF 1202 Silicone Fluid, a product corresponding to a decamethyl cyclopentasiloxane;

mixtures of two PDMS with different viscosities, for example, mixtures of a PDMS gum and a PDMS fluid, such as the SF 1236 product marketed by GENERAL ELECTRIC. The SF 1236 product is a mixture of a SE 30 gum such as defined hereabove with a viscosity of 20 m²/s and a SF 96 fluid with a viscosity of 5×10⁻⁶ m²/s. Such product may comprise 15% of a SE 30 gum and 85% of a SF 96 fluid.

The organopolysiloxane resins suitable for use according to the present disclosure include, but are not limited to, crosslinked siloxane systems comprising at least one of the following units:

### R₂SiO_{2/2}, R₃SiO_{1/2}, RSiO_{3/2}, and SiO_{4/2},

wherein R is an alkyl group comprising from 1 to 16 carbon atoms. According to at least one embodiment, R is a lower C₁-C₄ alkyl group, such as a methyl group.

These resins include, for example, the product marketed under the trade name “DOW CORNING 593” and those marketed under the trade names “SILICONE FLUID SS 4230 and SS 4267” by GENERAL ELECTRIC, that are dimethyl/trimethyl siloxane structured silicones.

Resins of the trimethyl siloxysilicate type may also be used, for instance, those marketed under the trade names X22-4914, X21-5034, and X21-5037 by SHIN-ETSU.

Polyalkylaryl siloxanes may be chosen from polydimethyl/methylphenyl siloxanes, linear and/or branched polydimethyl/diphenyl siloxanes with viscosities ranging from 1×0-5 to 5×10⁻² m²/s at 25° C.

Non-limiting examples of such polyalkylaryl siloxanes include the products marketed under the following trade names:

SILBIONE® fluids of the 70 641 series from RHODIA; RHODORSIL® fluids of the 70 633 and 763 series from RHODIA;

phenyl trimethicone fluid marketed under the reference DOW CORNING 556 COSMETIC GRADE FLUID by DOW CORNING;

PK series silicones from BAYER, for example, the PK20 product;

PN, PH series silicones from BAYER. for example, the PN1000 and PH1000 products; and

some SF series fluids from GENERAL ELECTRIC, such as SF 1023, SF 1154, SF 1250, and SF 1265.

Organomodified silicones which may be used according to the present disclosure include, but are not limited to, silicones such as those previously defined and comprising within their structure at least one organofunctional moiety linked by means of a hydrocarbon group.

Organomodified silicones may include, for example, polyorganosiloxanes comprising:

polyethyleneoxy and/or polypropyleneoxy moieties optionally comprising C₆-C₂₄ alkyl moieties, such as products called dimethicone copolyols marketed by DOW CORNING under the trade name DC 1248 and under the trade name DC Q2-5220 and SILWET® L 722, L 7500, L 77, and L 711 fluids marketed by UNION CARBIDE and the (C₁₂)alkyl-methicone copolyol marketed by DOW CORNING under the trade name Q2 5200;

optionally substituted amine moieties, for example, the products marketed under the trade name GP 4 Silicone Fluid and GP 7100 by GENESEE and the products marketed under the trade names Q2 8220 and DOW CORNING 929 and 939 by DOW CORNING. Substituted amine moieties may be chosen, for example, from amino C₁-C₄ alkyl moieties. Aminosilicones may have additional C₁-C₄ alkoxy functional groups, such as those corresponding to the WACKER BELSIL ADM LOG 1 product;

alkoxylated moieties, such as the product marketed under the trade name “SILICONE COPOLYMER F-755” by SWS SILICONES and ABIL WAX® 2428, 2434, and 2440 by GOLDSCHMIDT;

hydroxylated moieties, such as hydroxyalkyl function-containing polyorganosiloxanes described, for instance, in French Patent Application No. FR-A-85 163 34;

acyloxyalkyl moieties, for example, the polyorganosiloxanes described in U.S. Pat. No. 4,957,732;

anionic moieties of the carboxylic acid type, for example, the products described in European Patent No. 0 186 507, marketed by CHISSO CORPORATION, and carboxylic alkyl anionic moieties, such as those present in the X-22-3701E product marketed by SHIN-ETSU; 2-hydroxyalkyl sulfonate; and 2-hydroxyalkyl thiosulfate such as the products marketed by GOLDSCHMIDT under the trade names <<ABIL® S201>> and <<ABIL® S255>>;

hydroxyacylamino moieties, such as the polyorganosiloxanes described in European Patent Application No. 0 342 834. A non-limiting example of a corresponding commercial product is the Q2-8413 product marketed by DOW CORNING;

acrylic moieties, such as the products marketed under the names VS80 and VS70 by 3M;

polyamine moieties, and

oxazoline moieties

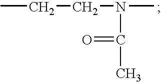

silicones that may be used according to the present disclosure may comprise 1 or 2 oxazoline groups; for example, poly(2-methyl oxazoline-b-dimethyl siloxane-b-2-methyl oxazoline) and poly(2-ethyl-2-oxazoline-dimethyl siloxane). The products marketed by KAO under the references OX-40, OS-51, OS-96, and OS-88 may also be used.

Suitable silicone-based celluloses which may be used according to the present disclosure include the products marketed by SHIN-ETSU under the references X-22-8401 and X-22-8404.

The at least one silicone used according to the present disclosure may be present in the composition in an amount ranging from 0.01 to 20%, for example, from 0.1 to 10%, such as from 0.1 to 5% by weight, relative to the total weight of the composition.

Solvents

The cosmetic composition according to the present disclosure typically comprises at least one solvent chosen from water, C₁-C₈ alcoholic solvents, and mixtures thereof.

C₁-C₈ alcoholic solvents may be chosen, by way of non-limiting example, from alkanols, alkane diols, benzyl alcohol, and phenylethyl alcohol.

According to one embodiment, the at least one solvent is chosen from ethanol, propanol, and isopropanol.

Additives

The cosmetic composition according to the present disclosure may also comprise at least one additional active cosmetic agent chosen, for example, from protein hydrolyzates, swelling and penetrating agents, agents for combating hair loss, anti-dandruff agents, non polymer synthetic and natural thickeners, suspending agents, sequestering agents, reducing agents, opacifying agents, dyes, sunscreen agents, vitamins and provitamins, fragrances and preserving agents, and pH regulating agents.

PH regulating agents may be chosen, for instance, from alkaline agents, such as ammonia, monoethanolamine, diethanolamine, triethanolamine, 1,3-propanediamine, alkaline hydroxides, such as 2-amino-2-methyl-1-propanol, and acidifying agents such as phosphoric acid and hydrochloric acid.

The pH of the composition of the present disclosure may range from 2 to 13, for example, from 4 to 11.

The at least one thickener may be chosen, for example, from cellulose-based thickeners, such as hydroxyethylcellulose, hydroxypropylcellulose, and carboxymethylcellulose, guar gum and derivatives thereof, for example, hydroxypropyl guar, marketed by RHODIA under the reference JAGUAR HP 105, microbial gums, such as xanthan gum and scleroglucan gum, synthetic thickeners such as cetyl stearyl alcohol, acrylic and acrylamidopropane sulfonic acid crosslinked homopolymers, for example Carbomer, associative non ionic, anionic, cationic, and amphoteric polymers, such as polymers marketed under the names PEMULEN TR1 and TR2 by GOODRICH, SALCARE SC90 by ALLIED COLLOIDS, ACULYN 22, 28, 33, 44, and 46 by ROHM & HAAS and ELFACOS T210 and T212 by AKZO.

Suitable conditioning agents which may be used according to the present disclosure include, but are not limited to, behentrimonium chloride marketed by CLARIANT under the reference GENAMIN KDNP.

The composition used according to the present disclosure may also comprise at least one organic acid.

The at least one organic acid may be chosen, for example, from acids comprising at least one functional group chosen from carboxylic, sulfonic, phosphonic, and phosphoric acid functional groups. They may further comprise other chemical functional groups, for instance, hydroxy and amino functional groups. They may be saturated or unsaturated.

Non-limiting examples of organic acids include acetic acid, propanoic acid, butanoic acid, lactic acid, glycolic acid, ascorbic acid, maleic acid, phthalic acid, succinic acid, taurine, tartaric acid, gluconic acid, glucuronic acid, and citric acid.

When at least one organic acid is present in the composition of the present disclosure, it is possible to increase the amount of water in the composition.

The composition used according to the present disclosure may be in any form that can suitably be applied onto the hair, for example, lotions, serum type solutions, gels, water-in-oil emulsions, oil-in-water emulsions, and combinations thereof, with a more or less thick, liquid consistency, such as milks and creams that are more or less creamy, and foams. Thus, the composition used according to the present disclosure may be in a form chosen from lotions, gels, foams, styling creams, treating creams, shampoos, after-shampoos, and colouring compositions.

The composition used according to the present disclosure may be conditioned in different forms, for example, in tubes, in cosmetic jars, in sprays, in pump containers, and in aerosol containers, in order to distribute the composition as a spray or as a foam.

When the composition is conditioned as an aerosol, it may be contained in a two-compartment container.

When the composition is conditioned in an aerosol device, it comprises at least one propellant, which may be chosen from volatile hydrocarbons, such as n-butane, propane, isobutane, pentane, halogenated hydrocarbons, and mixtures thereof. Additional examples of suitable propellants include, but are not limited to, carbon dioxide, nitrous oxide, dimethyl ether (DME), nitrogen, and compressed air. Combinations of propellants may also be used. According to at least one embodiment, the propellant is dimethyl ether.

The propellant may be present in the composition in an amount ranging from 5 to 90% by weight relative to the total weight of the composition within the aerosol device, for example, from 10 to 60%.

As discussed above, the composition of the present disclosure is a hair shaping composition.

For shaping the hair, the composition comprising the at least one alkoxysilane comprising at least one solubilizing functional group and the at least one silicone, is applied onto the hair, and optionally rinsed off. In at least one embodiment, the application is conducted by spraying, either via a pump bottle, or via an aerosol.

The composition of the present disclosure is then allowed to stand on the hair so as to let it penetrate into the hair, for example, for a time period of up to 30 minutes, such as from 5 to 15 minutes.

The hair may be rinsed with water after the composition is applied and allowed to react for a while.

The hair may then be set in form, for example, by means of a brush and/or an iron.

According to one embodiment, the hair is dried using a hairdryer and set in form with a brush.

During the hair drying, the at least one alkoxysilane comprising at least one solubilizing functional group and that are monomer compounds, are drying and polymerizing, thus forming materials that are neither soluble in water nor in the shampoo.

Once the hair has been set in form as previously described, it may look thicker, fuller, and more texturized, and may be easier to style.

The resulting styling effect is stronger than the one obtained with a hair shaping without applying the composition of the present disclosure.

The hair may be washed with shampoos, and then reshaped with brushing, and the same hair styling effects can be obtained. Such effects may last throughout several shampoos.

Also disclosed herein is a cosmetic composition comprising at least one silicone defined above and at least one alkoxysilane having at least one solubilizing functional group chosen from compounds of formulas (I-III).

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLES

Cosmetic compositions for hair shaping were formulated in accordance with the present disclosure.

### Example 1

### Styling Lotion 1

| Aminopropyl triethoxysilane (Dow Corning) | 10% | a.m.* |
|---|---|---|
| HCl | qs pH = 10.5 | |
| DC 939 Emulsion (Dow Corning) | 1% | a.m. |

| | |
|---|---|
| Demineralized water | qs 100% |

* “a.m.” indicates “active material.”

### Example 2

### Styling Lotion 2

| | | |
|---|---|---|
| 3-(m-aminophenoxy)propyl trimethoxysilane (Gelest) | 5% | a.m. |
| DC 1664 Emulsion (Dow Corning) | 1% | a.m. |
| Ethanol | 30% | a.m. |

| | |
|---|---|
| Demineralized water | qs 100% |

### Example 3

### Styling Lotion 3

| | | |
|---|---|---|
| Triethoxysilyl butyraldehyde (Gelest) | 2% | a.m. |
| DC 939 Emulsion (Dow Corning) | 1% | a.m. |
| Ethanol | 30% | a.m. |

| | |
|---|---|
| Demineralized water | qs 100% |

### Example 4

### Styling Lotion 4

| Aminopropyl triethoxysilane (Dow Corning) | 10% | a.m. |
|---|---|---|
| HCl | qs pH = 10.5. | |
| DC Q2-5220 Emulsion (Dow Corning) | 1% | a.m. |

| | |
|---|---|
| Demineralized water | qs 100% |

### Example 5

### Styling Gel

| Aminopropyl triethoxysilane (Dow Corning) | 10% | a.m. |
|---|---|---|
| HCl | qs pH = 10.5 | |
| DC 939 Emulsion (Dow Corning) | 1% | a.m. |
| Jaguar HP105 (Rhodia) | 1.5% | a.m. |

| | |
|---|---|
| Demineralized water | qs 100% |

### Example 6

### Styling Cream

| Aminopropyl triethoxysilane (Dow Corning) | 10% | a.m. |
|---|---|---|
| HCl | qs pH = 10.5 | |
| DC 939 Emulsion (Dow Corning) | 1% | a.m. |
| Cetestearyl alcohol | 2.5% | a.m. |
| Genamin KDMP (Clariant) | 0.4% | a.m. |

| | |
|---|---|
| Demineralized water | qs 100% |

### Example 7

### Strongly Treating Styling Lotion for Frizzy Hair

Part A:

| | | |
|---|---|---|
| Aminopropyl triethoxysilane (Dow Corning) | 10% | a.m. |

| | |
|---|---|
| HCl | qs pH = 10.5 |
| Demineralized water | qs 100% |

Part B:

| | | |
|---|---|---|
| Wacker Belsil ADM LOG1 (Wacker) | 2% | a.m. |

| | |
|---|---|
| Water | qs 100% |

Parts A and B were combined prior to application onto the hair.

The compositions were applied on wet hair.

Parts A and B may be also be allowed to react for 15 minutes, then the hair is rinsed off. The hair may be allowed to air dry, may be blown dry, or may be dried using flat clips.

The hair may also optionally dry without having been rinsed off. The hair may be allowed to air dry, may be blown dry, or may be dried using a flat clip iron.

Half-transient styling effects were thus obtained, as well as good cosmetic performances with regard to the feel of the hair, suppleness, and smoothness. These good performances were maintained even after a repeated application of such compositions.

What is claimed is:

1. A method for preventing the deterioration of the feel of the hair upon repeated application of a hair shaping composition comprising at least one alkoxysilane having at least one solubilizing functional group, the method comprising applying to the hair said hair shaping composition comprising at least one alkoxysilane comprising at least one solubilizing functional group, wherein said hair shaping composition further comprises at least one silicone.

2. The method of claim 1, wherein the at least one solubilizing functional group is chosen from primary, secondary, and tertiary amine, aromatic amine, alcohol, carboxylic acid, sulfonic acid, anhydride, carbamate, urea, guanidine, aldehyde, ester, amide, epoxy, pyrrole, dihydroimidazole, gluconamide, pyridyle, and polyether groups.

3. The method of claim 1, wherein the at least one alkoxysilane comprising at least one solubilizing functional group is chosen from compounds of formula (I): 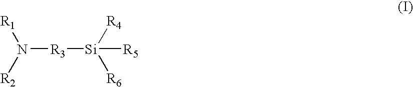
wherein:
R₄ is chosen from halogen atoms, OR′ groups, and R′₁ groups;
R₅ is chosen from halogen atoms, OR″ groups, and R′₂ groups;
R₆ is chosen from halogen atoms, OR′″ groups, and R′₃ groups;
R₁, R₂, R₃, R′, R″, R′″, R′₁, R′₂, and R′₃, which may be identical or different, are chosen from linear and branched, saturated and unsaturated hydrocarbon groups, optionally comprising at least one additional chemical group, wherein R₁, R₂, R′, R″, and R′″ may also be chosen from hydrogen, at least two groups R₄, R₅, and R₆ are different from R′₁, R′₂, and R′₃, and at least two groups R′, R″, and R′″ are not hydrogen.

4. The method of claim 3, wherein the R₁, R₂, R′, R′₁, R′₂, R′₃, R″, and R′″ groups are chosen from C₁-C₁₂ alkyl, C₆-C₁₄ aryl, C₁-C₈ alkyl- C₆-C₁₄₋ aryl, and C₆-C₁₄ aryl —C₁-C₈ alkyl radicals.

5. The method of claim 1, wherein the at least one alkoxysilane comprising at least one solubilizing functional group is chosen from compounds of formula (II): 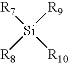
wherein:
R₉ is chosen from halogen atoms and OR′₉ groups and R₁₀ is chosen from halogen and OR′₁₀ groups; wherein at least one of R₉ and R₁₀ is not halogen;
R′₉ and R′₁₀, which may be identical or different, are chosen from hydrogen and linear and branched, saturated and unsaturated, C₁-C₁₄ hydrocarbon groups; wherein at least one of R′₉ and R′₁₀ is not hydrogen;
R₇ is a non hydrolyzable functional group providing a cosmetic effect, and
R₈ is a non hydrolyzable functional group bearing at least one function chosen from: amines, carboxylic acids and salts thereof, sulfonic acids and salts thereof, polyols, polyethers, and phosphoric acids and salts thereof.

6. The method of claim 1, wherein the at least one alkoxysilane comprising at least one solubilizing functional group is chosen from compounds of formula (III): 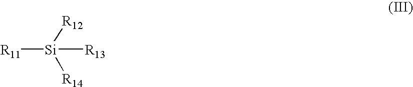
wherein:
R₁₂ is chosen from halogen atoms, OR′₁₂ groups, and R_{O} groups;
R₁₃ is chosen from halogen atoms, OR′₁₃ groups, and R′_{O} groups;
R₁₄ is chosen from halogen atoms, OR′₁₄ groups, and R″_{O} groups;
wherein at least two groups R₁₂, R₁₃, and R₁₄ are different from R_{O}, R′_{O}, and R″_{O};
R₁₁ is a group chosen from groups bearing at least one function chosen from: carboxylic acids and salts thereof, sulfonic acids and salts thereof, and polyalkylethers;
Ro, R′o, R″o, R′₁₂, R′₁₃, and R′₁₄, which may be identical or different, are chosen from linear and branched, saturated and unsaturated, C₁-C₁₄ hydrocarbon groups, optionally bearing at least one additional chemical function chosen from: carboxylic acids and salts thereof, sulfonic acids and salts thereof, and polyalkylether functional groups;
wherein R′₁₂, R′₁₃, and R₁₄ may also be chosen from hydrogen, and at least two groups R′₁₂, R′₁₃, and R′₁₄ are not hydrogen.

7. The method of claim 6, wherein R′₁₂, R′₁₃, R′₁₄, R_{O}, R′_{O}, and R″_{O}, which may be identical or different, are chosen from C₁-C₁₂ alkyl groups, C₆-C₁₄ aryl groups, C₁-C₈ alkyl-C₆-C₁₄ aryl groups, and C₆-C₁₄ aryl-C₁-C₈ alkyl groups

8. The method of claim 1, wherein the at least one alkoxysilane comprising at least one solubilizing functional group is chosen from compounds of formula (IV):
(R₂₁O)ₓ(R₂₂)_{y}Si-(A)ₚ-[NR₂₃-(A′)_{p′}]_{q}-[NR′₂₃-(A″)_{p″}]_{q′}-Si—(R′₂₂)_{y′}(OR′₂₁)_{x′} (IV)
wherein:
R₂₁, R₂₂, R′₂₁, and R′₂₂, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,
x is an integer ranging from 1 to 3,
y=3-x,
x′ is an integer ranging from 1 to 3,
y′=3-x′,
p=0 or 1,
p′=0 or 1,
p″=0 or 1,
q=0 or 1,
q′=0 or 1,
wherein at least one of q or q′ is not equal to zero,
A, A′, and A″, which may be identical or different, are chosen from linear and branched C₁-C₂₀ alkylene divalent radicals,
R₂₃ and R′₂₃, which may be identical or different, are chosen from hydrogen and linear and branched, saturated and unsaturated hydrocarbon chains, optionally comprising at least one heteroatom, optionally interrupted by or substituted with at least one entity chosen from ether, C₁-C₂₀ alcohol ester, amine, carboxyl, alkoxysilane, C₆-C₃₀ aryl, hydroxyl, and carbonyl groups, and aromatic, heterocyclic, and non-heterocyclic rings, optionally substituted with at least one group chosen from C₃-C₂₀ alcohol ester, amine, amide, carboxyl, alkoxysilane, hydroxyl, carbonyl, and acyl groups.

9. The method of claim 8, wherein:
R₂₁=R′₂₁,
R₂₂=R′₂₂,
x=x′,
y=y′,
p=p′,
A=A′,
q=1, and
q′=0.

10. The method of claim 8, wherein R₂₁, R₂₂, R′₂₁, and R′₂₂, which may be identical or different, are chosen from C₁-C₄ alkyl groups.

11. The method of claim 8, wherein p=p′=1.

12. The method of claim 8, wherein A and A′, which may be identical or different, are chosen from linear C₁-C₄ alkylene groups.

13. The method of claim 8, wherein R₂₃ is hydrogen.

14. The method of claim 1, wherein the at least one alkoxysilane comprising at least one solubilizing functional group is chosen from compounds of formula (V): 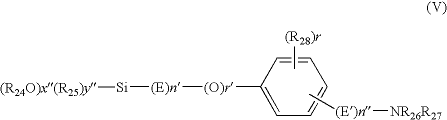
wherein:
R₂₄ and R₂₅, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,
x″=2 or 3,
y″=3-x″,
n′=0 or 1,
n″=0 or 1,
E and E′, which may be identical or different, are chosen from linear and branched C₁-C₂₀ alkylene divalent radicals,
R₂₆ and R₂₇, which may be identical or different, are chosen from hydrogen, and linear and branched, saturated and unsaturated hydrocarbon chains, optionally comprising at least one heteroatom, optionally interrupted by or substituted with at least one entity chosen from: ether, C₁-C₂₀ alcohol ester, amine, carboxyl, alkoxysilane, C₆-C₃₀ aryl, hydroxyl, and carbonyl groups, and aromatic, heterocyclic, and non heterocyclic rings, optionally substituted with at least one group chosen from C₁-C₂₀ alcohol ester, amine, amide, carboxyl, alkoxysilane, hydroxyl, carbonyl, and acyl groups,
r is an integer ranging from 0 to 4,
r′=0 or 1,
R₂₈, which may be identical or different is chosen from hydrogen and linear and branched, saturated and unsaturated hydrocarbon chains, optionally comprising at least one heteroatom, optionally interrupted by or substituted with at least one entity chosen from: ether, C₁-C₂₀ alcohol ester, amine, carboxyl, alkoxysilane, C₆-C₃₀ aryl, hydroxyl, and carbonyl groups, and aromatic, heterocyclic, and non-heterocyclic rings, optionally substituted with at least one group chosen from C₁-C₂₀ alcohol ester, amine, amide, carboxyl, alkoxysilane, hydroxyl, carbonyl, and acyl groups.

15. The method of claim 14, wherein R₂₄ is a C₁-C₄ alkyl group.

16. The method of claim 14, wherein x″=3.

17. The method of claim 14, wherein n′=n″=1.

18. The method of claim 14, wherein r=r′=0.

19. The method of claim 14, wherein R₂₆ and R₂₇, which may be identical or different, are chosen from hydrogen and groups chosen from C₁-C₄ alkyl groups, C₁-C₄ hydroxyalkyl groups, and C₁-C₄ aminoalkyl groups.

20. The method of claim 14, wherein the at least one alkoxysilane comprising at least one solubilizing functional group is chosen from:
3-(m-aminophenoxy)propyl trimethoxysilane, of formula: 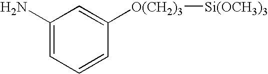
p-aminophenyl trimethoxysilane, of formula: 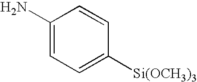
and
N-(2-aminoethylaminomethyl)phenethyl trimethoxysilane, of formula:

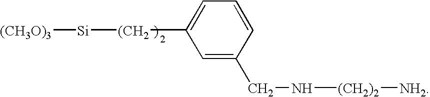

21. The method of claim 2, wherein the at least one alkoxysilane comprising at least one solubilizing functional group is chosen from compounds of formula (VI):
(R₂₉O)x₁(R₃₀)y₁-Si-(A₁)ₛ-CH═O (VI)
wherein:
R₂₉ and R₃₀, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,
x₁=2 or 3,
y₁=3-x₁,
A₁ is chosen from linear and branched C₁-C₂₀ alkylene divalent radicals, optionally interrupted by or substituted with at least one group chosen from C₁-C₃₀ alcohol ester, amine, carboxyl, alkoxysilane, C₆-C₃₀ aryl, hydroxyl, and carbonyl group, and
s=0 or 1.

22. The method of claim 21, wherein R₂₉ and R₃₀ are chosen from C₁-C₄ alkyl groups.

23. The method of claim 21, wherein s=1.

24. The method of claim 21, wherein A₁ is a linear C₁-C₄ alkylene group.

25. The method of claim 21, wherein the at least one alkoxysilane comprising at least one solubilizing functional group is chosen from:
triethoxysilyl butyraldehyde, of formula:
(CH₃CH₂O)₃—Si—(CH₂)₃—CH═O
triethoxysilyl undecanal, of formula:
(CH₃CH₂O)₃—Si—(CH₂)₁₀—CH═O
and
triethoxysilyl undecanal, ethylene glycol acetal, of formula:
(CH₃CH₂O)₃—Si—(CH₂)₁₀—CH(OCH₂)₂.

26. The method of claim 1, wherein the at least one alkoxysilane comprising at least one solubilizing functional group is chosen from compounds of formula (VII): 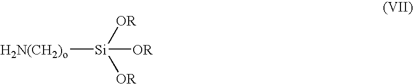
wherein R, which may be identical or different, is chosen from C₁-C₆ alkyl radicals and n is an integer ranging from 1 to 6.

27. The method of claim 26, wherein the at least one alkoxysilane comprising at least one solubilizing functional group is a γ-aminopropyl triethoxysilane.

28. The method of claim 1, wherein the at least one alkoxysilane comprising at least one solubilizing functional group is present in the composition in an amount ranging from 0.1 to 20% relative to the total weight of the composition.

29. The method of claim 1, wherein the at least one silicone is chosen from polydialkyl siloxanes, polyalkylaryl siloxanes, polydiaryl siloxanes, and organomodified polysiloxanes comprising at least one functional moiety chosen from poly(oxyalkylene) moieties, amine moieties, alkoxy moieties, hydroxylated moieties, acyloxyalkyl moieties, carboxylic acid moieties, hydroxyacylamino moieties, acrylic moieties, polyamine moieties, and oxazoline moieties, and silicone-based celluloses.

30. The method of claim 29, wherein the organomodified polysiloxanes are chosen from polydialkyl siloxanes that are organomodified with at least one moiety chosen from amine moieties, C₁-C₄ alkoxy moieties, carboxyl moieties, acrylic moieties, and polyamine moieties.

31. The method of claim 1, wherein the at least one silicone is present in the composition in an amount ranging from 0.01 to 20% by weight relative to the total weight of the composition.

32. The method of claim 1, wherein the composition comprises at least one solvent chosen from water and C₁-C₈ alcoholic solvents.

33. The method of claim 32, wherein the at least one C₁-C₈ alcoholic solvent is chosen from alkanols, alkane diols, benzyl alcohol, and phenyl ethyl alcohol.

34. The method of claim 33, wherein the at least one solvent is chosen from ethanol, propanol, and isopropanol.

35. The method of claim 1, wherein the hair shaping composition further comprises at least one cosmetic active agent chosen from protein hydrolyzates, swelling agents, penetrating agents, agents for combating hair loss, anti-dandruff agents, non polymer synthetic thickeners, non polymer natural thickeners, suspending agents, sequestering agents, reducing agents, opacifying agents, dyes, sunscreen agents, vitamins, provitamins, fragrances, preserving agents, pH regulating agents, and conditioning agents.

36. The method of claim 1, wherein the hair shaping composition further comprises at least one organic acid.

37. The method of claim 36, wherein the at least one organic acid is chosen from acetic acid, propanoic acid, butanoic acid, lactic acid, glycolic acid, ascorbic acid, maleic acid, phthalic acid, succinic acid, taurine, tartaric acid, gluconic acid, glucuronic acid, and citric acid.

38. A hair cosmetic composition comprising at least one silicone and at least one alkoxysilane comprising at least one solubilizing functional group, wherein the alkoxysilane comprising at least one solubilizing functional group is chosen from compounds of formula (I): 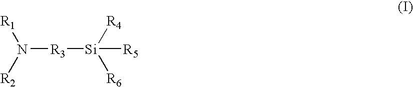
wherein:
R₄ is chosen from halogen atoms, OR′ groups, and R′₁ groups;
R₅ is chosen from halogen atoms, OR″ groups, and R′₂ groups;
R₆ is chosen from halogen atoms, OR′″ groups, and R′₃ groups;
R₁, R₂, R₃, R′, R″, R′″, R′₁, R′₂, and R′₃, which may be identical or different, are chosen from linear and branched, saturated and unsaturated hydrocarbon groups, optionally comprising at least one additional chemical group, wherein R₁, R₂, R′, R″, and R′″ may also be chosen from hydrogen, at least two groups R₄, R₅, and R₆ are different from R′₁, R′₂, and R′₃, and at least two groups R′, R″, and R′″ are not hydrogen.

39. A hair cosmetic composition comprising at least one silicone and at least one alkoxysilane having at least one solubilizing functional group, wherein the alkoxysilane comprising at least one solubilizing functional group is chosen from compounds of formula (II): 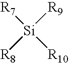
wherein:
R₉ is chosen from halogen atoms and OR′₉ groups and R₁₀ is chosen from halogen and OR′₁₀ groups; wherein at least one of R₉ and R₁₀ is not halogen;
R′₉ and R′₁₀, which may be identical or different, are chosen from hydrogen, and linear and branched, saturated and unsaturated, C₁-C₁₄ hydrocarbon groups; wherein at least one of R′₉ and R′₁₀ is not hydrogen;
R₇ is a non hydrolyzable functional group providing a cosmetic effect, and
R₈ is a non hydrolyzable functional group bearing at least one function chosen from: amines, carboxylic acids and salts thereof, sulfonic acids and salts thereof, polyols, polyethers, and phosphoric acids and salts thereof.

40. A hair cosmetic composition comprising at least one silicone and at least one alkoxysilane having at least one solubilizing functional group, wherein the alkoxysilane comprising at least one solubilizing functional group is chosen from compounds of formula (III): 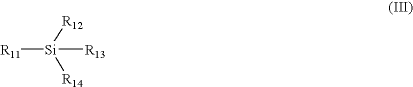
wherein:
R₁₂ is chosen from halogen atoms, OR′₁₂ groups, and R_{O} groups;
R₁₃ is chosen from halogen atoms, OR′₁₃ groups, and R′_{O} groups;
R₁₄ is chosen from halogen atoms, OR′₁₄ groups, and R″_{O} groups;
wherein at least two groups R₁₂, R₁₃, and R₁₄ are different from R_{O}, R′_{O}, and R″_{O};
R₁₁ is a group chosen from groups bearing at least one function chosen from: carboxylic acids and salts thereof, sulfonic acids and salts thereof, and polyalkylethers;
Ro, R′o, R″o, R′₁₂, R′₁₃, and R′₁₄, which may be identical or different, are chosen from linear and branched, saturated and unsaturated, C₁-C₁₄ hydrocarbon groups, optionally bearing at least one additional chemical function chosen from: carboxylic acids and salts thereof, sulfonic acids and salts thereof, and polyalkylether functions;
wherein R′₁₂, R′₁₃, and R₁₄ may also be chosen from hydrogen, and at least two groups R′₁₂, R′₁₃, and R′₁₄ are not hydrogen.

41. The cosmetic composition of claim 38, wherein the at least one silicone is chosen from polydialkyl siloxanes, polyalkylaryl siloxanes, polydiaryl siloxanes, and organomodified polysiloxanes comprising at least one functional moiety chosen from poly(oxyalkylene) moieties, amine moieties, alkoxy moieties, hydroxylated moieties, acyloxyalkyl moieties, carboxylic acid moieties, hydroxyacylamino moieties, acrylic moieties, polyamine moieties, oxazoline moieties, and silicone-based celluloses.

42. The composition of claim 41, wherein the organomodified polysiloxanes are chosen from polydialkyl siloxanes organomodified with at least one moiety chosen from amine moieties, C₁-C₄ alkoxy moieties, carboxyl moieties, acrylic moieties, and polyamine moieties.
